(19) United States
(12) Patent Application Publication
(10) Pub. No.: US 2020/0397310 A1
(43) Pub. Date: Dec. 24, 2020

(54) Smart-Device-Based Radar System Detecting Human Vital Signs in the Presence of Body Motion

(71) Applicant: Google LLC, Mountain View, CA (US)

(72) Inventors: Changzhan Gu, Milpitas, CA (US); Jaime Lien, Mountain View, CA (US); Jian Wang, Cupertino, CA (US)

(73) Assignee: Google LLC, Mountain View, CA (US)

(21) Appl. No.: 16/957,991

(22) PCT Filed: Feb. 28, 2019
(86) PCT No.: PCT/US2019/020022

§ 371 Date: Jun. 25, 2020

(51) Int. Cl. A61B 5/0205; G01S 7/41; A61B 5/00; A61B 5/11

(52) U.S. Cl. CPC A61B 5/0205; G01S 7/417; G01S 7/415; A61B 5/7207; G01S 13/584; A61B 5/1114; A61B 5/1126; A61B 5/1118; A61B 5/7278; A61B 5/7267

(57) ABSTRACT

Techniques and apparatuses are described that implement a smart-device-based radar system capable of detecting human vital signs in the presence of body motion. In particular, a radar system includes a body-motion filter module that employs machine learning to filter body motion from a received radar signal and construct a filtered signal that includes information regarding a user's vital signs. With machine learning, the radar system can filter the body motion without relying on data from other sensors to determine the body motion. Furthermore, the body-motion filter module can be trained to compensate for a variety of different types of body motions, such as those that occur while a user sleeps, exercises, drives, works, or is treated by a medical professional. By filtering the body motion, the radar system can accurately determine the user's vital signs and provide non-contact human vital-sign detection.

### BACKGROUND

A health monitoring device can help a user improve or maintain their health by measuring and reporting the user's vital signs. With this information, the health monitoring device can track a user's progress towards a fitness goal or provide notification of a detected anomaly to enable the user to quickly obtain medical attention. Some health monitoring devices, however, are obtrusive and require contact with the user's skin to accurately measure the user's vital signs. This may make it cumbersome for the user to use throughout the day or impede actions of nurses or doctors that are tending to the user.

To address this problem, some health monitoring devices utilize a radar sensor to provide contactless health monitoring. There are many challenges associated with detecting human vital signs using a radar sensor, however. One such challenge involves detecting a user's vital signs in the presence of unintentional or intentional body motions from the user or another nearby person. The body motions can interfere with the radar sensor's ability to detect the user's vital sign. Consequently, vital-sign detection performance of the radar sensor can degrade in the presence of one or more types of body motion.

### SUMMARY

Techniques and apparatuses are described that implement a smart-device-based radar system capable of detecting human vital signs in the presence of body motion. In particular, a radar system includes a body-motion filter module that employs machine learning to filter the body motion from a received radar signal and construct a filtered signal that includes information regarding a user's vital signs, such as a heart rate or a respiration rate. With machine learning, the radar system can recognize and extract the body motion without relying on data from other sensors, such as a camera or another radar system, to determine the body motion. In other words, the proposed machine learning technique enables a single radar system to detect human vital signs in the presence of body movement compared to other techniques that utilize multiple radar systems or sensor fusion to compensate for the body movement. As such, hardware modifications and increased hardware complexity or cost can be avoided. Furthermore, the body-motion filter module can be trained to compensate for a variety of different types of body motions, such as those that occur while a user sleeps, exercises, drives, works, or is treated by a medical professional. By filtering the body motion, the radar system can accurately determine the user's vital signs and provide non-contact vital-sign detection.

Aspects described below include an apparatus with a radar system. The radar system includes at least one antenna, a transceiver, a body-motion filter module, and a vital-sign detection module. The transceiver is coupled to the at least one antenna and is configured to transmit, via the at least one antenna, a radar transmit signal. The transceiver is also configured to receive, via the at least one antenna, a radar receive signal. The radar receive signal includes a portion of the radar transmit signal that is reflected by a user. The radar receive signal also includes a superposition of a vital-sign component signal and a body-motion component signal. The vital-sign component signal is associated with at least one vital sign of the user and the body-motion component signal is associated with at least one motion of the user. The body-motion filter module is coupled to the transceiver and is configured to accept an input data sequence associated with the radar receive signal. Additionally, the body-motion filter module is configured to filter, using machine learning, the body-motion component signal from the input data sequence to produce a filtered data sequence based on the vital-sign component signal. The vital-sign detection module is coupled to the body-motion filter module and is configured to determine the at least one vital sign of the user based on the filtered data sequence.

Aspects described below also include a method for performing operations of a smart-device-based radar system capable of detecting human vital signs in the presence of body motion. The method includes transmitting a radar transmit signal and receiving a radar receive signal. The radar receive signal includes a portion of the radar transmit signal that is reflected by a user. Additionally, the radar receive signal includes a superposition of a vital-sign component signal and a body-motion component signal. The vital-sign component signal is associated with at least one vital sign of the user and the body-motion component signal is associated with at least one motion of the user. The method also includes generating an input data sequence based on the radar receive signal. Using a machine-learned module, the method includes filtering the body-motion component signal from the input data sequence to produce a filtered data sequence based on the vital-sign component signal. The method further includes determining the at least one vital sign of the user based on the filtered data sequence.

Aspects described below also include a computer-readable storage media comprising computer-executable instructions that, responsive to execution by a processor, implement a body-motion filter module and a vital-sign detection module. The body-motion filter module is configured to accept a first input data sequence associated with a first radar receive signal. The first radar receive signal includes a superposition of a first vital-sign component signal and a first body-motion component signal. The first vital-sign component signal is associated with at least one first vital sign of a user and the first body-motion component signal is associated with at least one first body motion of the user. The body-motion filter module is also configured to filter, using machine learning, the first body-motion component signal from the first input data sequence to produce a first filtered data sequence based on the first vital-sign component signal. The vital-sign detection module is configured to determine the at least one first vital sign of the user based on the first filtered data sequence.

Aspects described below also include a system with machine-learning means for filtering body motion from a radar receive signal to determine a user's vital signs.

### BRIEF DESCRIPTION OF THE DRAWINGS

Apparatuses for and techniques implementing a smart-device-based radar system capable of detecting human vital signs in the presence of body motion are described with reference to the following drawings. The same numbers are used throughout the drawings to reference like features and components:

FIG. 1 illustrates example environments in which a smart-device-based radar system capable of detecting human vital signs in the presence of body motion can be implemented.

FIG. 2 illustrates an example implementation of a radar system as part of a smart device.

FIG. 3 illustrates an example operation of a radar system for detecting human vital signs in the presence of body motion.

FIG. 4 illustrates an example scheme performed by a body-motion filter module for detecting human vital signs in the presence of body motion.

FIG. 5 illustrates an example implementation of a machine-learned module for detecting human vital signs in the presence of body motion.

FIG. 6 illustrates an example method for performing operations of a smart-device-based radar system capable of detecting human vital signs in the presence of body motion.

FIG. 7 illustrates an example computing system embodying, or in which techniques may be implemented that enable use of, a radar system capable of detecting human vital signs in the presence of body motion.

### DETAILED DESCRIPTION

Overview

There are many challenges associated with detecting vital signs using a radar sensor. One such challenge involves detecting a user's vital signs in the presence of unintentional or intentional body motion of the user or another nearby person. This body motion can interfere with the radar sensor's ability to detect the user's vital sign. Consequently, vital-sign detection performance of the radar sensor can degrade in the presence of one or more types of body motion.

Some radar-based health monitoring devices utilize multiple radar sensors and restrain a user between the multiple radar sensors to limit body motion. The use of multiple radar sensors, however, increases a complexity and cost of the health monitoring device. Furthermore, the process of restraining the user can be cumbersome and time consuming. Other radar-based health monitoring devices incorporate an optical sensor, such as a camera, to measure the body motion and provide the radar sensor information regarding the body motion. The addition of the camera, however, can also increase complexity, cost, and size of the radar-based health monitoring device.

In contrast, techniques described herein present a smart-device-based radar system capable of detecting human vital signs in the presence of body motion. In particular, a radar system includes a body-motion filter module that employs machine learning to filter the body motion from a received radar signal and construct a filtered signal that includes information regarding a user's vital signs, such as a heart rate or a respiration rate. With machine learning, the radar system can recognize and extract the body motion without relying on data from other sensors, such as a camera or another radar system, to determine the body motion. In other words, the proposed machine learning technique enables a single radar system to detect human vital signs in the presence of body movement compared to other techniques that utilize multiple radar systems or sensor fusion to compensate for the body movement. As such, hardware modifications and increased hardware complexity or cost can be avoided. Furthermore, the body-motion filter module can be trained to compensate for a variety of different types of body motions, such as those that occur while a user sleeps, exercises, drives, works, or is treated by a medical professional. By filtering the body motion, the radar system can accurately determine the user's vital signs and provide non-contact vital-sign detection.

Example Environment

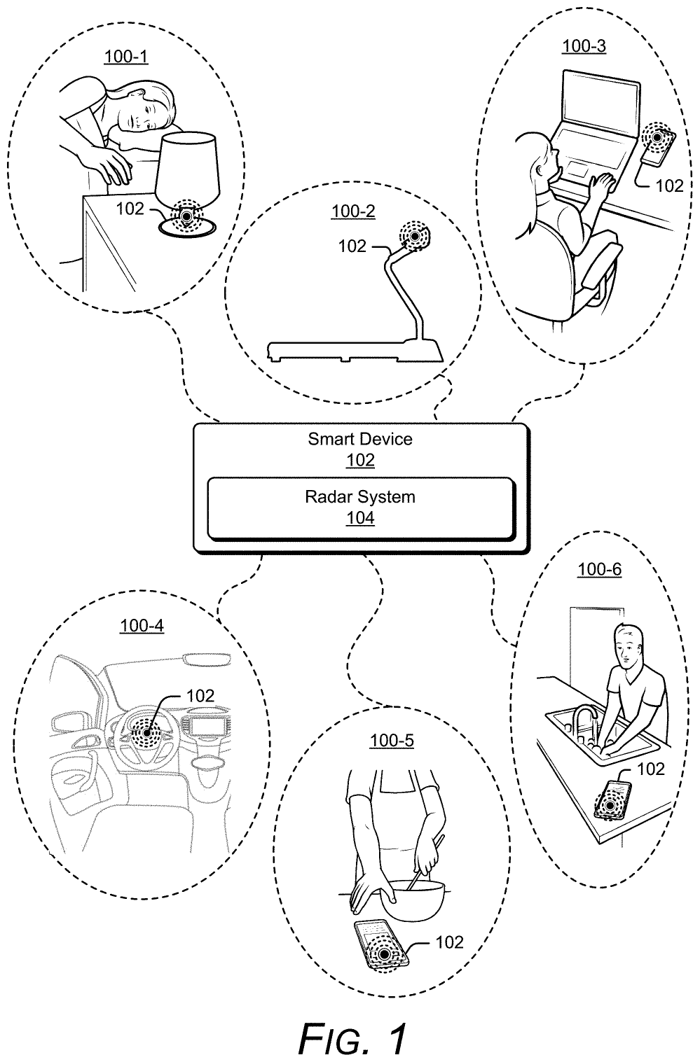

FIG. 1 is an illustration of example environments in which techniques using, and an apparatus including, a smart-device-based radar system capable of detecting human vital signs in the presence of body motion may be embodied. In the depicted environments 100-1, 100-2, 100-3, 100-4, 100-5, and 100-6, a smart device 102 includes a radar system 104 capable of providing non-contact vital-sign detection. The smart device 102 is shown to be a lamp in the environment 100-1, a treadmill in the environment 100-2, a smart phone in the environments 100-3, 100-5, and 100-6, and a steering wheel in the environment 100-4.

A variety of different types of body motions can occur while the user performs an activity. While sleeping in the environment 100-1, the user may, for example, rotate their body to change sleeping positions or move their arm to adjust a pillow. In the environment 100-2, the user may move up and down while exercising on the treadmill and move to different positions on the treadmill that are closer to or farther from the radar system 104 as they change their speed. While the user works at a desk in the environment 100-3, the user may rock or rotate in the chair, move their arm to control a mouse or touch pad, or move their arm to reach for an item on the desk. In the environment 100-4, the user drives a vehicle and may move their arms to steer the vehicle, change gears, or adjust a thermostat. While preparing a meal in the environment 100-5 or washing dishes in the environment 100-6, the user may move their arms in front of their bodies to mix food in a bowl or clean a plate. In other environments not shown, the user may walk around a room or another person may move portions of their body between the radar system 104 and the user.

To provide non-contact vital-sign detection in the presence of body motion, the radar system 104 uses machine learning to recognize and filter these different types of body motions from a radar receive signal. In this manner, the smart device 102 can use a single radar system for non-contact vital-sign detection and does not need to incorporate other types of sensors to determine the user's body motion. Furthermore, by filtering the body motion, an accuracy of the radar system 104 increases for determining the user's vital signs in the presence of body motion. Although described within the context of human vital-sign detection, the techniques described can also be used to detect vital signs of animals.

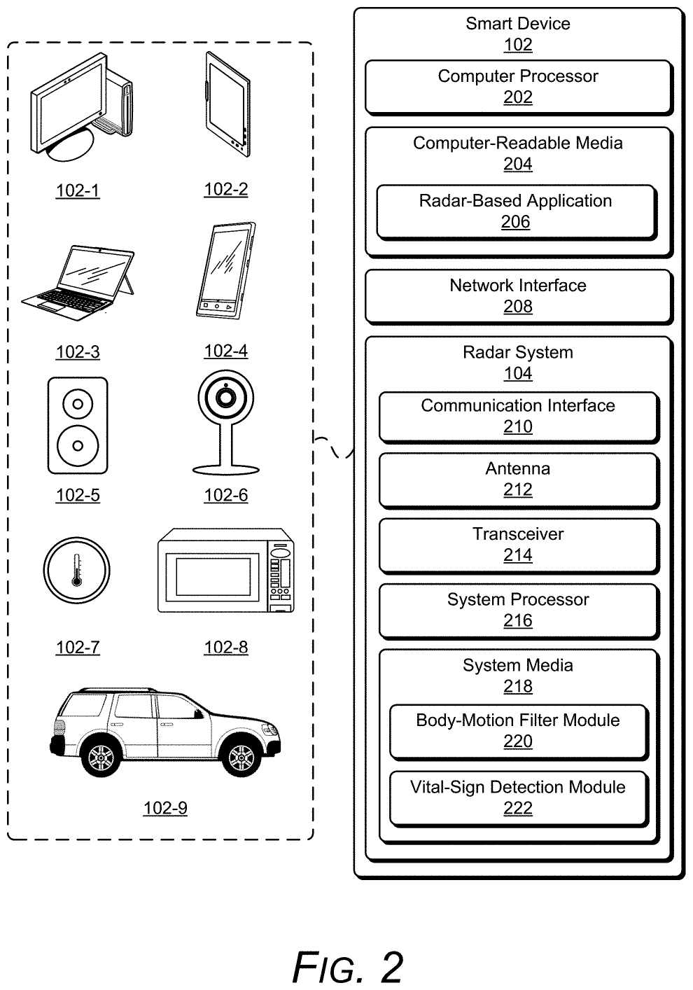

Some implementations of the radar system 104 are particularly advantageous as applied in the context of human vital-sign health monitoring systems, for which there is a convergence of issues such as a need for limitations in a spacing and layout of the radar system 104, low power, and other issues. Although the implementations are particularly advantageous in the described context of a system for which human vital-sign detection and monitoring is required, it is to be appreciated that the applicability of the features and advantages of the present invention is not necessarily so limited, and other implementations involving other types of electronic devices may also be within the scope of the present teachings. Although the smart device 102 is shown as different household or vehicle objects in FIG. 1, the smart device 102 can be implemented as any suitable computing or electronic device, as described in further detail with respect to FIG. 2.

Exemplary overall lateral dimensions of the smart device 102 can be, for example, approximately eight centimeters by approximately fifteen centimeters. Exemplary footprints of the radar system 104 can be even more limited, such as approximately four millimeters by six millimeters with antennas included. Exemplary power consumption of the radar system 104 may be on the order of a few milliwatts to several milliwatts (e.g., between approximately two milliwatts and twenty milliwatts). The requirement of such a limited footprint and power consumption for the radar system 104, enables the smart device 102 to include other desirable features in such a space-limited package (e.g., a camera sensor, a fingerprint sensor, a display, and so forth). The smart device 102 and the radar system 104 are further described with respect to FIG. 2.

FIG. 2 illustrates the radar system 104 as part of the smart device 102. The smart device 102 can be any suitable computing device or electronic device, such as a desktop computer 102-1, a tablet 102-2, a laptop 102-3, a smartphone 102-4, a smart speaker 102-5, a security camera 102-6, a smart thermostat 102-7, a microwave 102-8, and a vehicle 102-9. Other devices may also be used, such as home-service devices, baby monitors, routers, computing watches, computing glasses, gaming systems, televisions, drones, track pads, drawing pads, netbooks, e-readers, home-automation and control systems, and other home appliances. The smart device 102 can be wearable, non-wearable but mobile, or relatively immobile (e.g., desktops and appliances). The radar system 104 can be used as a stand-alone radar system or used with, or embedded within, many different computing devices or peripherals, such as in control panels that control home appliances and systems, in automobiles to control internal functions (e.g., volume, cruise control, or even driving of the car), or as an attachment to a laptop computer to control computing applications on the laptop.

The smart device 102 includes one or more computer processors 202 and computer-readable media 204, which includes memory media and storage media. Applications and/or an operating system (not shown) embodied as computer-readable instructions on the computer-readable media 204 can be executed by the computer processor 202 to provide some of the functionalities described herein. The computer-readable media 204 also includes a radar-based application 206, which uses radar data generated by the radar system 104 to perform a function, such as human vital-sign notification, gesture-based control, presence detection, or collision avoidance for autonomous driving.

The smart device 102 also includes a network interface 208 for communicating data over wired, wireless, or optical networks. For example, the network interface 208 communicates data over a local-area-network (LAN), a wireless local-area-network (WLAN), a personal-area-network (PAN), a wire-area-network (WAN), an intranet, the Internet, a peer-to-peer network, a point-to-point network, a mesh network, and the like. The smart device 102 may also include a display or speakers (not shown).

The radar system 104 includes a communication interface 210 to transmit the radar data to a remote device, though this need not be used if the radar system 104 is integrated within the smart device 102. In general, the radar data provided by the communication interface 210 is in a format usable by the radar-based application 206.

The radar system 104 also includes at least one antenna 212 and at least one transceiver 214 to transmit and receive radar signals. The antenna 212 can be circularly polarized, horizontally polarized, or vertically polarized. In some cases, the radar system 104 includes multiple antennas 212 implemented as antenna elements of an antenna array. The antenna array can include at least one transmitting antenna element and at least two receiving antenna elements. In some situations, the antenna array includes multiple transmitting antenna elements to implement a multiple-input multiple-output (MIMO) radar capable of transmitting multiple distinct waveforms at a given time (e.g., a different waveform per transmitting antenna element). The receiving antenna elements can be positioned in a one-dimensional shape (e.g., a line) or a two-dimensional shape (e.g., a triangle, a rectangle, or an L-shape) for implementations that include three or more receiving antenna elements. The one-dimensional shape enables the radar system 104 to measure one angular dimension (e.g., an azimuth or an elevation) while the two-dimensional shape enables two angular dimensions to be measured (e.g., both azimuth and elevation).

Using the antenna array, the radar system 104 can form beams that are steered or un-steered, wide or narrow, or shaped (e.g., as a hemisphere, cube, fan, cone, or cylinder). The one or more transmitting antenna elements may have an un-steered omnidirectional radiation pattern or may be able to produce a wide steerable beam. Either of these techniques enable the radar system 104 to illuminate a large volume of space. To achieve target angular accuracies and angular resolutions, the receiving antenna elements can be used to generate thousands of narrow steered beams (e.g., 2000 beams, 4000 beams, or 6000 beams) with digital beamforming. In this way, the radar system 104 can efficiently monitor an external environment and detect vital signs from one or more users.

The transceiver 214 includes circuitry and logic for transmitting and receiving radar signals via the antenna 212. Components of the transceiver 214 can include amplifiers, mixers, switches, analog-to-digital converters, filters, and so forth for conditioning the radar signals. The transceiver 214 also includes logic to perform in-phase/quadrature (I/Q) operations, such as modulation or demodulation. A variety of modulations can be used to produce the radar signals, including linear frequency modulations, triangular frequency modulations, stepped frequency modulations, or phase modulations. Alternatively, the transceiver 214 can produce radar signals having a relatively constant frequency or a single tone. The transceiver 214 can be configured to support continuous-wave or pulsed radar operations.

A frequency spectrum (e.g., range of frequencies) that the transceiver 214 can use to generate radar signals can encompass frequencies between 1 and 400 gigahertz (GHz), between 1 and 24 GHz, between 2 and 4 GHz, between 4 and 100 GHz, between 57 and 63 GHz, or at approximately 2.4 GHz. In some cases, the frequency spectrum can be divided into multiple sub-spectrums that have similar or different bandwidths. Example bandwidths can be on the order of 500 megahertz (MHz), one gigahertz (GHz), two gigahertz, and so forth. Different frequency sub-spectrums may include, for example, frequencies between approximately 57 and 59 GHz, 59 and 61 GHz, or 61 and 63 GHz. Although the example frequency sub-spectrums described above are contiguous, other frequency sub-spectrums may not be contiguous. To achieve coherence, multiple frequency sub-spectrums (contiguous or not) that have a same bandwidth may be used by the transceiver 214 to generate multiple radar signals, which are transmitted simultaneously or separated in time. In some situations, multiple contiguous frequency sub-spectrums may be used to transmit a single radar signal, thereby enabling the radar signal to have a wide bandwidth.

The radar system 104 may also include one or more system processors 216 and a system media 218 (e.g., one or more computer-readable storage media). Although the system processor 216 is shown to be separate from the transceiver 214 in FIG. 2, the system processor 216 may be implemented within the transceiver 214 as a digital signal processor or a low-power processor, for instance. The system processor 216 executes computer-readable instructions that are stored within the system media 218. Example digital operations performed by the system processor 216 include Fast-Fourier Transforms (FFTs), filtering, modulations or demodulations, digital signal generation, digital beamforming, and so forth.

The system media 218 includes a body-motion filter module 220 and a vital-sign detection module 222 (e.g., a human vital-sign detection module 222). The body-motion filter module 220 employs machine learning to filter (e.g., cancel or extract) the body motion from a received radar signal and construct a filtered signal that includes information regarding a user's vital signs. In other words, the body-motion filter module 220 effectively attenuates a portion of a received radar signal that corresponds to the body motion and passes another portion of the received radar signal that corresponds to the user's vital signs. Using machine learning, the body-motion filter module 220 reconstructs an amplitude and frequency of the received radar signal in a manner that substantially removes disturbances caused by the body motion and substantially includes disturbances associated with the user's vital signs to produce a filtered signal.

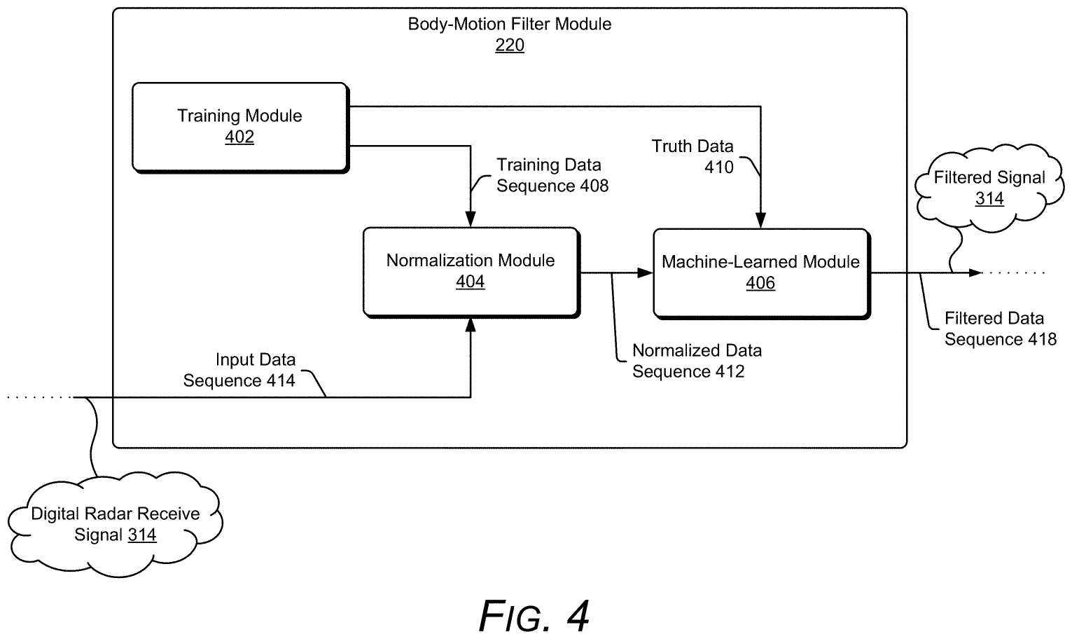

The body-motion filter module 220 relies on supervised learning and can use simulated (e.g., synthetic) data or measured (e.g., real) data for machine-learning training purposes, as further described with respect to FIG. 4. In some implementations, the smart device 102 includes a contact-based sensor (not shown), which generates truth data by measuring the user's vital signs while in contact with the user's skin. This training enables the body-motion filter module 220 to learn a non-linear mapping function for translating a radar receive signal, which includes interference from body motion as well as information about the user's vital signs, into a filtered signal that includes the information about the user's vital signs.

The body-motion filter module 220 can include one or more artificial neural networks (referred to herein as neural networks). A neural network includes a group of connected nodes (e.g., neurons or perceptrons), which are organized into one or more layers. As an example, the body-motion filter module 220 includes a deep neural network, which includes an input layer, an output layer, and one or more hidden layers positioned between the input layer and the output layers. The nodes of the deep neural network can be partially-connected or fully connected between the layers.

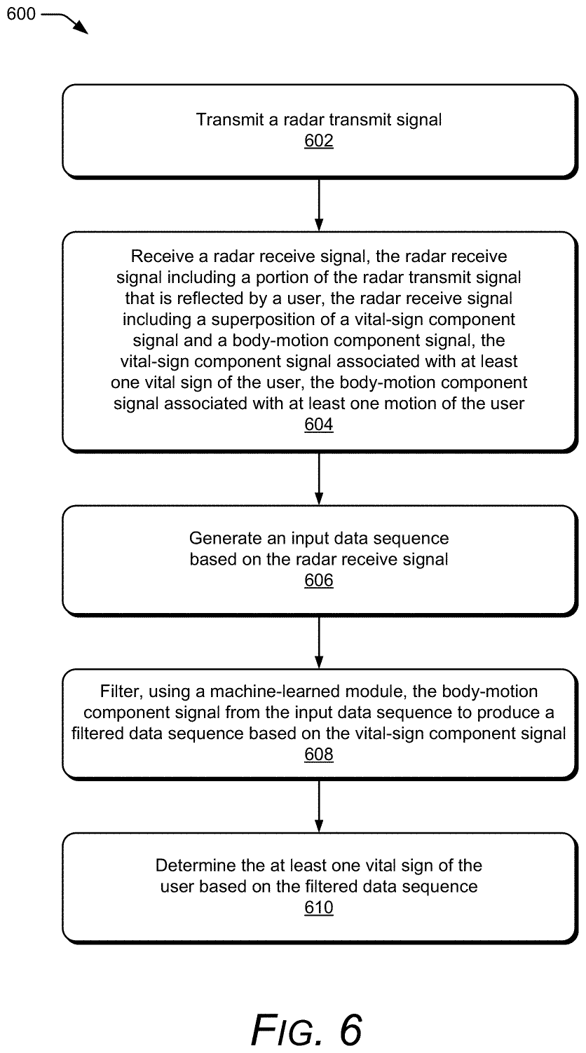

In some cases, the deep neural network is a recurrent deep neural network (e.g., a long short-term memory (LSTM) recurrent deep neural network) with connections between nodes forming a cycle to retain information from a previous portion of an input data sequence for a subsequent portion of the input data sequence. In other cases, the deep neural network is a feed-forward deep neural network in which the connections between the nodes do not form a cycle. Additionally or alternatively, the body-motion filter module 220 can include another type of neural network, such as a convolutional neural network. An example deep neural network is further described with respect to FIG. 6.

The body-motion filter module 220 can also include one or more types of regression models, such as a single linear regression model, multiple linear regression models, logistic regression models, step-wise regression models, multi-variate adaptive regression splines, locally estimated scatterplot smoothing models, and so forth.

Generally, a machine learning architecture of the body-motion filter module 220 can be tailored based on available power, available memory, or computational capability. The machine learning architecture can also be tailored based on a quantity of body motions or a complexity of the body motions the body-motion filter module 220 is designed to filter. In some cases, the body-motion filter module 220 can be trained to automatically filter body motions associated with a variety of different activities. In this way, the radar system 104 can seamlessly provide non-contact vital-sign detection as a user switches between different activities.

Alternatively, to reduce a complexity of the body-motion filter module 220, the body-motion filter module 220 can be re-trained for each activity a user performs. In this case, the radar-based application 206 can prompt the user to select a particular activity and inform the body-motion filter module 220 of the selected activity for training purposes. By using machine learning, the body-motion filter module 220 can filter the body motion without receiving additional data from other sensors that measure the body motion.

The vital-sign detection module 222 receives the filtered signal from the body-motion filter module 220 and analyzes the filtered signal to determine the user's vital signs, such as the user's heart rate or respiration rate. The vital-sign detection module 222 can use the communication interface 210 to inform the radar-based application 206 of the user's vital signs. The radar-based application 206 can monitor the user's vital signs to detect an anomaly or communicate the vital sign measurements to the user via the display or speakers of the smart device 102.

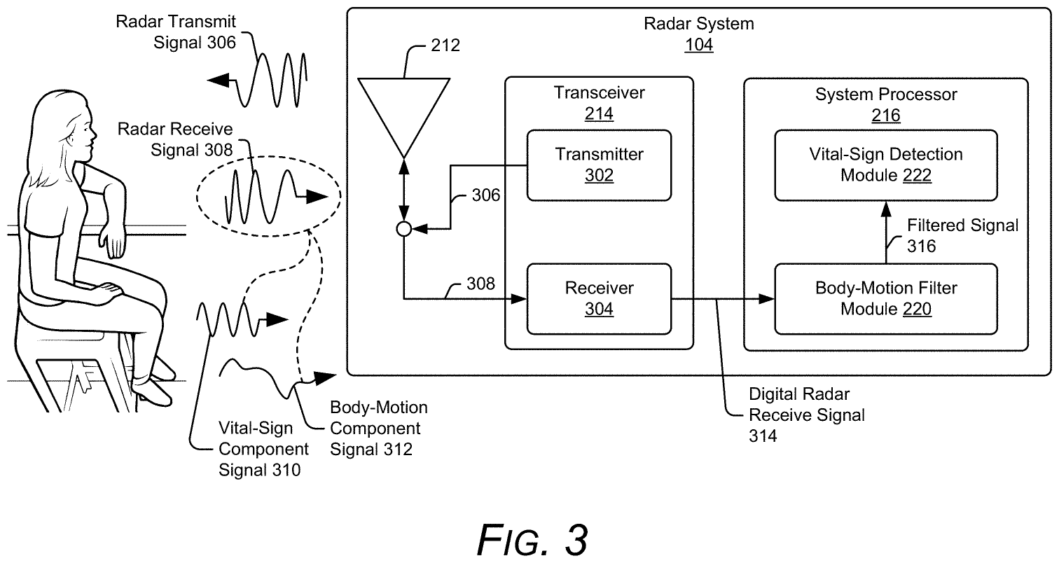

Although shown to be included within the system media 218, other implementations of the body-motion filter module 220 and/or the vital-sign detection module 222 can be included, at least partially, within the computer-readable media 204. In this case, at least some functionality of the body-motion filter module 220 or the vital-sign detection module 222 can be by the computer processor 202. Although not shown, the system media 218 can also include other types of modules, such as a gesture recognition module, a collision avoidance module, a user detection module, a digital beamforming module, and so forth. The radar system 104 is further described with respect to FIG. 3.

Detecting Human Vital Signs in the Presence of Body Motion

FIG. 3 illustrates an example operation of the radar system 104 for detecting human vital signs in the presence of body motion. In the depicted configuration, the radar system 104 is shown to include the antenna 212, the transceiver 214, and the system processor 216. The antenna 212 is indirectly or directly coupled to the transceiver 214, which includes a transmitter 302 and a receiver 304. The system processor 216 is coupled to the transceiver 214 and executes the body-motion filter module 220 and the vital-sign detection module 222.

During operation, the transmitter 302 generates and provides a radar transmit signal 306 to the antenna 212. In some cases, the radar transmit signal 306 is a frequency-modulated signal with a frequency that varies over time, as shown in FIG. 3. In other cases, the radar transmit signal 306 is a continuous-sinusoidal signal that has a relatively steady (e.g., approximately constant) frequency.

The antenna 212 transmits the radar transmit signal 306, which impinges on a user. Consequently, a radar receive signal 308 is reflected from the user and includes at least a portion of the radar transmit signal 306. Due to the Doppler effect, however, a frequency of the radar receive signal 308 differs from the radar transmit signal 306 based on the user's vital signs and body motion. More specifically, the radar receive signal 308 includes a superposition of a vital-sign component signal 310 and a body-motion component signal 312. The vital-sign component signal 310 includes amplitude and frequency information associated with the user's vital signs, such as the user's heart rate and the user's respiration rate. In contrast, the body-motion component signal 312 includes amplitude and frequency information associated with the user's body motions (or body motions of another nearby person). The body-motion component signal 312 causes the amplitude and frequency of the radar receive signal 308 to fluctuate. These fluctuations can make it challenging for the radar system 104 to accurately measure the user's vital signs directly from the radar receive signal 308 (e.g., without filtering or compensating for the body-motion component signal 312).

The receiver 304 receives the radar receive signal 308 via the antenna 212 and generates a digital radar receive signal 314 based on the radar receive signal 308. For example, the receiver 304 downconverts the radar receive signal 308 to a baseband frequency and samples the downconverted signal to produce the digital radar receive signal 314. The sampling rate of the receiver 304 can be based on a predicted frequency range of probable vital-sign component signals to avoid aliasing. The digital radar receive signal 314 includes a temporal sequence of samples of the radar receive signal 308, which are provided as an input data sequence to the body-motion filter module 220, as shown in FIG. 4.

The body-motion filter module 220 generates a filtered signal 316 based on the digital radar receive signal 314. In particular, the body-motion filter module 220 processes different sets of samples based on a temporal processing window, filters the body-motion component signal 312 within these sets of samples, and outputs sets of filtered samples that are associated with the vital-sign component signal 310. In effect, the body-motion filter module 220 compensates for amplitude and/or frequency disturbances within the radar receive signal 308 resulting from the body-motion component signal 312 and produces the filtered signal 316 based on the vital-sign component signal 310.

A size of the temporal processing window, and therefore a quantity of samples within each set of samples, can be predetermined based on a predicted temporal stability of the vital-sign component signal 310. In this manner, the body-motion filter module 220 operates under an assumption that an amplitude and frequency of the vital-sign component signal 310 is relatively stable throughout a duration of the temporal processing window. The body-motion filter module 220 provides the filtered signal 316 to the vital-sign detection module 222. Although not explicitly shown, the receiver 304 or the system processor 216 can also include a band-pass filter that filters the radar receive signal 308 for frequencies outside a general frequency range of the vital-sign component signal 310 prior to providing the digital radar receive signal 314 to the body-motion filter module 220.

The vital-sign detection module 222 determines the user's vital signs based on the filtered signal 316. Since the user's heart rate and respiration rate typically have different frequency ranges, the vital-sign detection module 222 filters and extracts the different frequencies associated with the heart rate and respiration rate from the filtered signal 316. As an example, the user's heart rate can be at approximately 65 beats per minute while the user's respiration rate can be at approximately 18 breaths per minute. The vital-sign detection module 222 can also perform an FFT to identify frequency peaks that respectively correspond to the heart rate and the respiration rate. Although not explicitly shown, the vital-sign detection module 222 can inform the radar-based application 206 of the determined vital signs of the user. Operations performed by the body-motion filter module 220 are further described with respect to FIG. 4.

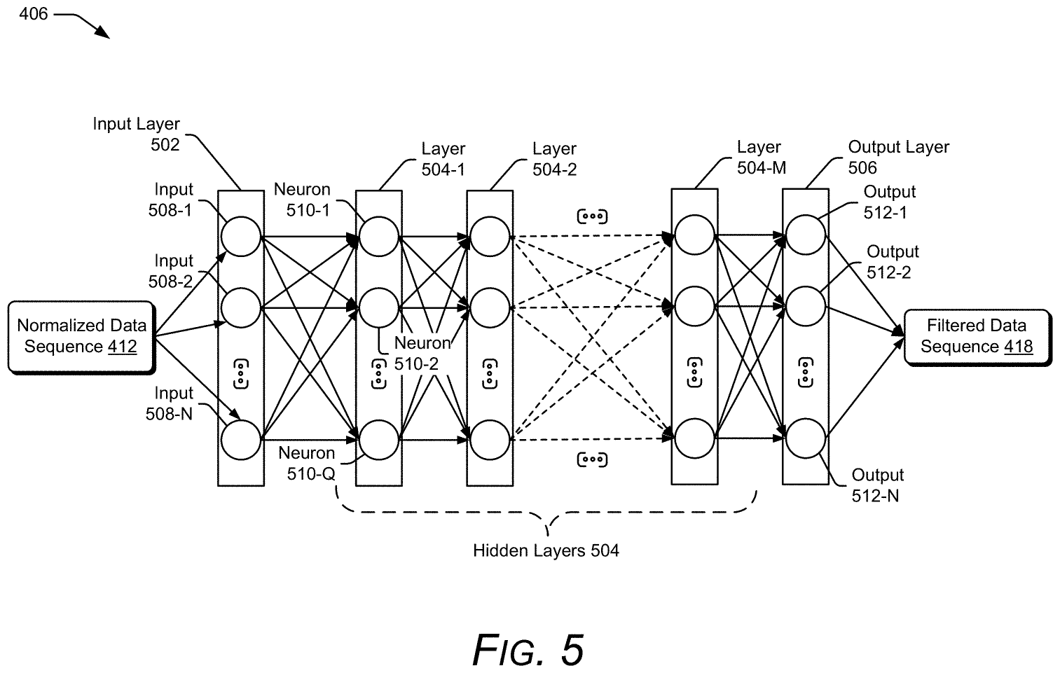

FIG. 4 illustrates an example scheme performed by the body-motion filter module 220 for human vital-sign detection in the presence of body motion. In the depicted configuration, the body-motion filter module 220 includes a training module 402, a normalization module 404, and a machine-learned module 406. In general, the machine-learned module 406 can be implemented using one or more of the machine learning architectures described above with respect to FIG. 2. An example implementation of the machine-learned module 406 is further described with respect to FIG. 5.

The training module 402 is coupled to the normalization module 404 and the machine-learned module 406. The normalization module 404 is also coupled to an input of the body-motion filter module 220, which can be coupled to the receiver 304 (of FIG. 3). The machine-learned model 406 is coupled to the normalization module 404 and an output of the body-motion filter module 220, which can be coupled to the vital-sign detection module 222, as shown in FIG. 3.

The training module 402 provides a training data sequence 408 and truth data 410 for training the machine-learned module 406 to filter body motions associated with one or more activities. The training data sequence 408 and the truth data 410 can be based on simulated data or measured data, either of which can be stored within the system media 218 or generated in real time during an initialization procedure. Although the training module 402 is shown to be included within the body-motion filter module 220 in FIG. 4, the training module 402 can alternatively be implemented separate from the body-motion filter module 220.

In the simulated data case, the training module 402 generates sinusoidal signals to simulate probable vital-sign component signals that represent different heart rates, respiration rates, or a combination thereof. The sinusoidal signals can be periodic signals and vary in frequency and/or phase from each other. The truth data 410 includes the sinusoidal signals, which the training module 402 provides to the machine-learned module 406 during a training procedure.

Additionally, the training module 402 generates perturbation signals to simulate probable body-motion component signals. In some cases, the perturbation signals represent different types of body motions associated with a particular activity, such as different types of motions of a user's arm, different rotations of the user's body about at least one first axis, different translations of the user's body across at least one second axis, a combination thereof, and so forth. The training module 402 can include a random number generator or a body-motion simulator to generate different perturbation signals. The random number generator generates random samples, which simulate different amplitude and/or frequency fluctuations that can result from relatively simple body motions, such as translation-type motions. In contrast, the body-motion simulator includes algorithms that simulate a series of amplitude and/or frequency fluctuations that result from more complex body motions, such as different types of motions of a user's arm. Using the samples generated via the random number generator or via the body-motion simulator, the training module 402 performs an interpolation operation, such as a shape-preserving piecewise cubic interpolation operation, to interpolate between the samples. Based on the interpolation, the training module 402 performs a down-sampling operation to produce samples of the perturbation signal. In general, the sinusoidal signals and the perturbation signals are generated to have a similar quantity of samples.

The training module 402 sums different pairs of the sinusoidal signals and the perturbation signals together to synthesize the training data sequence 408. In this manner, the training data sequence 408 includes superpositions of different probable vital-sign component signals with different probable body-motion component signals. A quantity of paired sinusoidal signals and perturbation signals can vary based on a complexity of the body motion, and can be on the order of thousands to hundreds of thousands of pairs, for instance. During the training procedure, the training module 402 provides the training data sequence 408 to the normalization module 404, as shown in FIG. 4, or to the machine-learned module 406 if the training data sequence 408 is normalized.

In the measured data case, the training module 402 can be coupled to a contact-based sensor within smart device 102, which generates the truth data 410 by measuring the user's vital signs while in contact with the user's skin (e.g., while the user touches the smart device 102). The training module 402 receives the truth data 410 from the contact-based sensor and passes the truth data 410 to the machine-learned module 406 during the training procedure. Additionally, the training module 402 is coupled to the transceiver 214 of FIG. 3, and causes the radar system 104 to operate (e.g., transmit one or more radar transmit signals 306 and receive one or more radar receive signals 308) during a time period that the contact-based sensor generates the truth data 410. In this way, any motion performed by the user during this time period is captured by the radar receive signals 308. The training module 402 can perform an extrapolation operation to generate the training data sequence 408 based on the radar receive signal 308. The training procedure is further described below.

The normalization module 404 performs a normalization operation that generates a normalized data sequence 412 based on an input signal (e.g., an input data sequence 414 or the training data sequence 408). As one example, the normalization module 404 can normalize the input signal by subtracting a mean value of the input signal across a given dimension's feature values from each individual feature value and then dividing by the standard deviation or another metric. By normalizing the input signal, the body-motion filter module 220 is able to account for amplitude variations resulting from changes in a user's distance from the radar system 104 during non-contact vital-sign detection. This normalization operation also enables the machine-learned module 406 to efficiently determine weights and bias parameters that optimize a cost function (e.g., an objective function).

During a training procedure, the training module 402 provides a training data sequence 408 to the normalization module 404 and associated truth data 410 to the machine-learned module 406. The normalization module 404 normalizes the training data sequence 408 and provides a normalized data sequence 412 to the machine-learned module 406. The machine-learned module 406 processes the normalized data sequence 412 and generates a filtered data sequence 418. The machine-learned module 406 also determines weights and bias parameters that minimize an error between the resulting filtered data sequence 418 and the truth data 410 using a cost function, such as a mean square error. As an example, the machine-learned module 406 can use a gradient descent method to optimize the cost function. Generally speaking, this training procedure enables the machine-learned module 406 to effectively filter the body-motion component signal 312 and generate the filtered data sequence 418 based on the vital-sign component signal 310.

During non-contact vital-sign detection, the normalization module 404 accepts the input data sequence 414 from an input of the body-motion filter module 220. As described with respect to FIG. 3, this input data sequence 414 can represent the digital radar receive signal 314, which is provided by the receiver 304. The normalization module 404 normalizes the digital radar receive signal 314 and provides the normalized data sequence 412 to the machine-learned module 406. Using the weights and bias parameters determined during the training procedure, the machine-learned module filters the body component signal 312 from the normalized data sequence 412 and generates the filtered data sequence 418 based on the vital-sign component signal 310. The machine-learned module 406 is further described with respect to FIG. 5.

FIG. 5 illustrates an example implementation of the machine-learned module 406 for human vital-sign detection in the presence of body motion. In the depicted configuration, the machine-learned module 406 is implemented as a deep neural network and includes an input layer 502, multiple hidden layers 504, and an output layer 506. The input layer 502 includes multiple inputs 508-1, 508-2 . . . 508-N, where N represents a positive integer equal to a quantity of samples corresponding to the temporal processing window. The multiple hidden layers 504 include layers 504-1, 504-2 . . . 504-M, where M represents a positive integer. Each hidden layer 504 includes multiple neurons, such as neurons 510-1, 510-2 . . . 510-Q, where Q represents a positive integer. Each neuron 510 is connected to at least one other neuron 510 in a previous hidden layer 504 or a next hidden layer 504. A quantity of neurons 510 can be similar or different between different hidden layers 504. In some cases, a hidden layer 504 can be a replica of a previous layer (e.g., layer 504-2 can be a replica of layer 504-1). The output layer 506 includes outputs 512-1, 512-2 . . . 512-N.

Generally speaking, a variety of different deep neural networks can be implemented with various quantities of inputs 508, hidden layers 504, neurons 510, and outputs 512. A quantity of layers within the machine-learned module 406 can be based on the quantity of body motions and the complexity of the body motion that the body-motion filter module 220 is designed to filter. As an example, the machine-learned module 406 can include four layers (e.g., one input layer 502, one output layer 506, and two hidden layers 504) to filter rocking motions that a user makes in a chain (e.g., such as in the example environment 100-3 of FIG. 1). Alternatively, the quantity of hidden layers can be on the order of a hundred to enable the body-motion filter module 220 to filter a variety of different arm motions a user makes while sleeping, exercising, driving, preparing a meal, or washing dishes, as described with respect to environments 100-1, 100-2, 100-4, 100-5, and 100-6.

During vital-sign detection, a set of input samples associated with the normalized data sequence 412 is provided to the input layer 502 based on the temporal processing window. Assuming the digital radar receive signal 314 is generated based on a sampling rate of 20 Hz and a size of the temporal processing window represents a duration of 4 seconds, the set of input samples includes 80 samples, and a quantity of inputs 508 and outputs 512 (e.g., N) is equal to 80. Each neuron 510 in the hidden layers 504 analyzes a different section or portion of the set of input samples for different features. Together, the hidden layers 504 compensate for disturbances that are present within the digital radar receive signal 314 based on the body-motion component signal 312. At the output layer 506, a set of filtered samples is generated, which is based on the vital-sign component signal 310. The vital-sign detection module 222 can analyze the set of filtered samples to determine the user's vital signs during this time period.

The above operations can continue for a subsequent set of input samples within the normalized data sequence 412. With training, the machine-learned module 406 can learn to filter a variety of different types of body motions to enable non-contact vital-sign detection to be performed while the user participates in a variety of different activities.

Example Methods

FIG. 6 depicts an example method 600 for performing operations of a smart-device-based radar system capable of detecting human vital signs in the presence of body motion. Method 600 is shown as sets of operations (or acts) performed but not necessarily limited to the order or combinations in which the operations are shown herein. Further, any of one or more of the operations may be repeated, combined, reorganized, or linked to provide a wide array of additional and/or alternate methods. In portions of the following discussion, reference may be made to the environment 100-1 to 100-6 of FIG. 1, and entities detailed in FIG. 2 or 4, reference to which is made for example only. The techniques are not limited to performance by one entity or multiple entities operating on one device.

At 602, a radar transmit signal is transmitted. For example, the radar system 104 transmits the radar transmit signal 306 using the transmitter 302 and the antenna 212, as shown in FIG. 3. In different implementations, the radar transmit signal 306 can be a frequency-modulated signal (e.g., a chirp signal) or a sinusoidal signal with a relatively constant frequency, and a continuous-wave signal or a pulsed signal.

At 604, a radar receive signal is received. The radar receive signal includes a portion of the radar transmit signal that is reflected by a user, and includes a superposition of a vital-sign component signal and a body-motion component signal. The vital-sign component signal is associated with at least one vital sign of the user and the body-motion component signal is associated with at least one motion of the user.

For example, the radar system 104 receives the radar receive signal 308 using the receiver 304 and the antenna 212, as shown in FIG. 3. The radar receive signal 308 includes a portion of the radar transmit signal 306 that is reflected by the user, such as a user shown in the example environments 100-1 to 100-6 of FIG. 1. The radar receive signal 308 also includes a superposition of the vital-sign component signal 310 and the body-motion component signal 312. The vital-sign component signal 310 is associated with one or more vital signs of the user, such as a heart rate or a respiration rate. In contrast, the body-motion component signal 312 is associated with one or more motions of the user, such as a motion of the user's appendage (e.g., an arm or a leg), a rotation of the user's body, a translation of the user's body, or a combination thereof. Alternatively or additionally, the body-motion component signal 312 is associated with a motion of another person that is nearby the user. In this case, the radar receive signal 308 includes another portion of the radar transmit signal 306 that is reflected by this nearby person.

At 606, an input data sequence is generated based on the radar receive signal. For example, the receiver 304 generates the digital radar receive signal 314 by downconverting and sampling the radar receive signal 308. The sampling rate of the receiver 304 can be based on a frequency range of probable vital-sign component signals to avoid aliasing, and can be on the order of tens of hertz, for instance. Additionally, the normalization module 404 of the body-motion filter module 222 can normalize the input data sequence 414 to generate the normalized data sequence 412, as shown in FIG. 4. This normalization process accounts for amplitude variations resulting from the user being at different distances from the radar system 104 during non-contact vital-sign detection.

At 608, the body-motion component signal is filtered from the input data sequence using a machine-learned module to produce a filtered data sequence based on the vital-sign component signal. For example, the body-motion filter module 220 filters the body-motion component signal 312 from the input data sequence 414 (or the normalized data sequence 412) using the machine-learned module 406 and produces the filtered data sequence 418 based on the vital-sign component signal 310. The machine-learned module 406 can be a deep neural network that is trained to recognize and extract a variety of different types of body-motion components signals associated with one or more user activities.

At 610, the at least one vital sign of the user is determined based on the filtered data sequence. For example, the vital-sign detection module 222 determines the at least one vital sign of the user based on the filtered data sequence 418. The vital-sign detection module 222 can further provide the determined vital sign to the radar-based application 206, which communicates the measured vital sign to the user.

Example Computing System

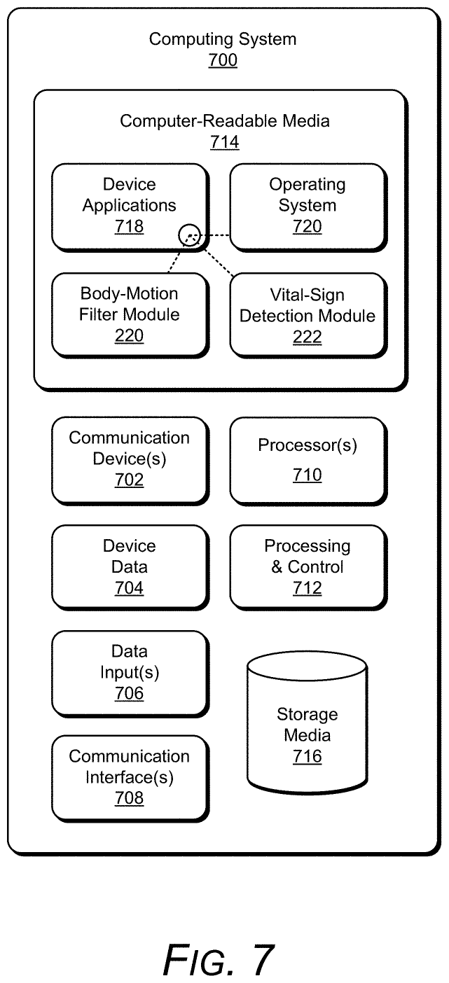

FIG. 7 illustrates various components of an example computing system 700 that can be implemented as any type of client, server, and/or computing device as described with reference to the previous FIG. 2 to implement human vital-sign detection in the presence of body motion.

The computing system 700 includes communication devices 702 that enable wired and/or wireless communication of device data 704 (e.g., received data, data that is being received, data scheduled for broadcast, or data packets of the data). The device data 704 or other device content can include configuration settings of the device, media content stored on the device, and/or information associated with a user of the device. Media content stored on the computing system 700 can include any type of audio, video, and/or image data. The computing system 700 includes one or more data inputs 706 via which any type of data, media content, and/or inputs can be received, such as human utterances, user-selectable inputs (explicit or implicit), messages, music, television media content, recorded video content, and any other type of audio, video, and/or image data received from any content and/or data source.

The computing system 700 also includes communication interfaces 708, which can be implemented as any one or more of a serial and/or parallel interface, a wireless interface, any type of network interface, a modem, and as any other type of communication interface. The communication interfaces 708 provide a connection and/or communication links between the computing system 700 and a communication network by which other electronic, computing, and communication devices communicate data with the computing system 700.

The computing system 700 includes one or more processors 710 (e.g., any of microprocessors, controllers, and the like), which process various computer-executable instructions to control the operation of the computing system 700 and to enable techniques for, or in which can be embodied, human vital-sign detection in the presence of body motion. Alternatively or in addition, the computing system 700 can be implemented with any one or combination of hardware, firmware, or fixed logic circuitry that is implemented in connection with processing and control circuits which are generally identified at 712. Although not shown, the computing system 700 can include a system bus or data transfer system that couples the various components within the device. A system bus can include any one or combination of different bus structures, such as a memory bus or memory controller, a peripheral bus, a universal serial bus, and/or a processor or local bus that utilizes any of a variety of bus architectures.

The computing system 700 also includes a computer-readable media 714, such as one or more memory devices that enable persistent and/or non-transitory data storage (i.e., in contrast to mere signal transmission), examples of which include random access memory (RAM), non-volatile memory (e.g., any one or more of a read-only memory (ROM), flash memory, EPROM, EEPROM, etc.), and a disk storage device. The disk storage device may be implemented as any type of magnetic or optical storage device, such as a hard disk drive, a recordable and/or rewriteable compact disc (CD), any type of a digital versatile disc (DVD), and the like. The computing system 700 can also include a mass storage media device (storage media) 716.

The computer-readable media 714 provides data storage mechanisms to store the device data 704, as well as various device applications 718 and any other types of information and/or data related to operational aspects of the computing system 700. For example, an operating system 720 can be maintained as a computer application with the computer-readable media 714 and executed on the processors 710. The device applications 718 may include a device manager, such as any form of a control application, software application, signal-processing and control module, code that is native to a particular device, a hardware abstraction layer for a particular device, and so on.

The device applications 718 also include any system components, engines, or managers to implement human vital-sign detection in the presence of body motion. In this example, the device applications 718 include the body-motion filter module 220 and the vital-sign detection module 222.

### CONCLUSION

Although techniques using, and apparatuses including a smart-device-based radar system detecting human vital signs in the presence of body motion have been described in language specific to features and/or methods, it is to be understood that the subject of the appended claims is not necessarily limited to the specific features or methods described. Rather, the specific features and methods are disclosed as example implementations of smart-device-based radar system detecting human vital signs in the presence of body motion.

1. A smart device comprising:
a radar system, the radar system including:
at least one antenna;
a transceiver coupled to the at least one antenna and configured to:
transmit, via the at least one antenna, a radar transmit signal;
receive, via the at least one antenna, a radar receive signal, the radar receive signal including a portion of the radar transmit signal that is reflected by a user, the radar receive signal including a superposition of a vital-sign component signal and a body-motion component signal, the vital-sign component signal associated with at least one vital sign of the user, the body-motion component signal associated with at least one motion of the user; and
generate an input data sequence based on the radar receive signal, the input data sequence comprising a temporal sequence of digital samples of the radar receive signal;
a body-motion filter module coupled to the transceiver and configured to:
accept the input data sequence associated with the radar receive signal; and
filter, using machine learning, the body-motion component signal from the input data sequence to produce a filtered data sequence based on the vital-sign component signal; and
a vital-sign detection module coupled to the body-motion filter module and configured to determine the at least one vital sign of the user based on the filtered data sequence.

2. The smart device of claim 1, wherein the body-motion filter module includes a normalization module coupled to the transceiver, the normalization module configured to normalize the input data sequence to produce a normalized data sequence.

3. The smart device of claim 2, wherein the body-motion filter module includes a machine-learned module coupled to the normalization module, the machine-learned module configured to:
accept a set of normalized samples within the normalized data sequence based on a temporal processing window, a size of the temporal processing window based on a predetermined temporal stability of the at least one vital sign of the user; and
filter the body-motion component signal from the set of normalized samples to produce a set of filtered samples associated with the filtered data sequence, the set of normalized samples and the set of filtered samples having similar quantities of samples based on the size of the temporal processing window.

4. The smart device of claim 3, wherein:
the body-motion filter module includes a training module coupled to the machine learned module and the normalization module, the training module configured to:
provide a training data sequence to the normalization module; and
provide truth data to the machine-learned module;
the normalization module is configured to generate another normalized data sequence based on the training data sequence; and
the machine-learned module is configured to execute a training procedure to determine machine-learning parameters based on the other normalized data sequence and the truth data.

5. The smart device of claim 4, wherein the training module is configured to:
generate sinusoidal signals to simulate probable vital-sign component signals, the sinusoidal signals representing the truth data;
generate perturbation signals using a random number generator to simulate probable body-motion component signals; and
combine different pairs of the sinusoidal signals and the perturbation signals together to generate the training data sequence.

6. The smart device of claim 5, wherein:
the sinusoidal signals are periodic; and
the sinusoidal signals differ in phase or frequency.

7. The smart device of claim 4, further comprising:
a sensor configured to generate the truth data by measuring the at least one vital sign of the user through contact with the user's skin,
wherein the training module is coupled to the sensor and configured to:
pass the truth data from the sensor to the machine-learned module;
cause the radar system to transmit at least one other radar transmit signal and receive at least one other radar receive signal while the sensor generates the truth data; and
generate the training data sequence based on the at least one other radar receive signal.

8. The smart device of claim 1, wherein the body-motion filter module includes a machine-learned module comprising a deep neural network with at least two hidden layers.

9. The smart device of claim 1, further comprising:
a radar-based application configured to communicate a heart rate and a respiration rate to the user,
wherein the at least one vital sign of the user comprises the heart rate and the respiration rate.

10. The smart device of claim 1, wherein the at least one motion of the user comprises at least one of the following:
a motion of the user's arm;
a rotation of the user's body about at least one first axis; or
a translation of the user's body across at least one second axis.

11. The smart device of claim 1, wherein:
the radar receive signal includes another portion of the radar transmit signal that is reflected by a person that is near the user; and
the body-motion component signal is associated with the at least one motion of the user and the at least one other motion of the person.

12. A method comprising:
transmitting a radar transmit signal;
receiving a radar receive signal, the radar receive signal including a portion of the radar transmit signal that is reflected by a user, the radar receive signal including a superposition of a vital-sign component signal and a body-motion component signal, the vital-sign component signal associated with at least one vital sign of the user, the body-motion component signal associated with at least one motion of the user;
generating an input data sequence based on the radar receive signal, the input data sequence comprising a temporal sequence of digital samples of the radar receive signal;
filtering, using a machine-learned module, the body-motion component signal from the input data sequence to produce a filtered data sequence based on the vital-sign component signal; and
determining the at least one vital sign of the user based on the filtered data sequence.

13. The method of claim 12, further comprising:
prompting the user to select an activity from a list of activities, the list of activities including a first activity;
determining that a first selection of the user corresponds to the first activity; and
training the machine-learned module to filter probable body-motion component signals associated with the first activity.

14. The method of claim 13, further comprising:
prompting the user to select another activity from the list of activities, the list of activities including a second activity;
determining that a second selection of the user corresponds to the second activity; and
training the machine-learned module to filter other probable body-motion component signals associated with the second activity.

15. The method of claim 13, wherein the training of the machine-learned module comprises:
generating sinusoidal signals to simulate probable vital-sign component signals;
providing the sinusoidal signals as truth data to the machine-learned module;
generating perturbation signals using a random number generator to simulate the probable body-motion component signals;
combining different pairs of the sinusoidal signals and the perturbation signals together to generate a training data sequence; and
providing the training data sequence to the machine-learned module.

16. The method of claim 13, wherein the training of the machine-learned module comprises:
obtaining measurement data associated with the at least one vital sign of the user from a contact-based sensor during a given time period;
transmitting at least one other radar transmit signal during the given time period;
receiving at least one other radar receive signal associated with the at least one other radar transmit signal during the given time period;
generating truth data based on the measurement data;
generating a training data sequence based on the at least one radar receive signal; and
providing the training data sequence and the truth data to the machine-learned module.

17. A computer-readable storage media comprising computer-executable instructions that, responsive to execution by a processor, implement:
a body-motion filter module configured to:
accept a first input data sequence comprising a temporal sequence of digital samples of a first radar receive signal, the first radar receive signal includes a superposition of a first vital-sign component signal and a first body-motion component signal, the first vital-sign component signal associated with at least one first vital sign of a user, the first body-motion component signal associated with at least one first body motion of the user; and
filter, using machine learning, the first body-motion component signal from the first input data sequence to produce a first filtered data sequence based on the first vital-sign component signal; and
a vital-sign detection module configured to determine the at least one first vital sign of the user based on the first filtered data sequence.

18. The computer-readable storage media of claim 17, wherein the body-motion filter module includes:
a normalization module configured to normalize the input data sequence to produce a normalized data sequence; and
a machine-learned module configured to:
accept a set of normalized samples within the normalized data sequence based on a temporal processing window, a size of the temporal processing window based on a predetermined temporal stability of the at least one vital sign of the user; and
filter the body-motion component signal from the set of normalized samples to produce a set of filtered samples associated with the filtered data sequence, the set of normalized samples and the set of filtered samples having similar quantities of samples based on the size of the temporal processing window.

19. The computer-readable storage media of claim 17, wherein:
the body-motion filter module is configured to:
execute a training procedure to enable filtering of a second body-motion component signal, the second body-motion component signal associated with at least one second motion of the user;
accept a second input data sequence associated with a second radar receive signal, the second radar receive signal comprising another superposition of a second vital-sign component signal and the second body-motion component signal, the second vital-sign component signal associated with at least one second vital sign of the user; and
filter, using the machine learning, the second body-motion component signal from the second input data sequence to produce a second filtered data sequence based on the second vital-sign component signal; and
the vital-sign detection module is configured to determine the at least one second vital sign of the user based on the second filtered data sequence.

20. The computer-readable storage media of claim 19, wherein the computer-executable instructions, responsive to execution by the processor, implement a radar-based application configured to communicate the at least one first vital sign and the at least one second vital sign to the user.
